(19) United States
(12) Patent Application Publication
(10) Pub. No.: US 2006/0018883 A1
(43) Pub. Date: Jan. 26, 2006

(54) Microbial preparation & method for preventing and curing the bacterial wilt the plant and its use

(76) Inventors: Yuanguang Li, Shanghai (CN); Wei Wang, Shanghai (CN); Honggang Wei, Shanghai (CN); Guomin Shen, Shanghai (CN); Rongjin Wang, Shanghai (CN); Jialu Deng, Shanghai (CN); Xinzhi Sun, Shanghai (CN); Ming Shen, Shanghai (CN)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 10/537,419

(22) PCT Filed: Dec. 1, 2003
(86) PCT No.: PCT/CN03/01025

§ 371 Date: Jun. 2, 2005

(30) Foreign Application Priority Data
Dec. 4, 2002 (CN) 0215019.9

(51) Int. Cl. A01N 63/00; C12N 1/20

(52) U.S. Cl. 424/93.4; 435/252.3

(57) ABSTRACT

Formulations comprising bacterial of non-toxigenic strain Paenibacillus polymyxa or the cell-free filtrate obtained from its culture broth are provided as biological agents for control of plant pathogens. Particularly, the formulation of this invention is effective not only in inhibiting bacterial wilt and other plant diseases such as seedling Rhizoctonia, damping off (Pythium aphanidermatum), tomato Fusarium wilt, eggplant Fusarium wilt, cucumber Fusarium wilt, Tobacco brown leaf spot (Alternaria altelnata) and Soybean Fusarium root rot (Fusarium orthocreras), but also in promoting plant growth.

### FIELD OF INVENTION

The present invention relates to a biocontrol formulation effective in inhibiting bacterial wilt, its application method and biocontrol functions.

### DESCRIPTION OF THE PRIOR ART

Bacterial wilt, caused by R. solanacearum, is a worldly, soil-borne plant disease, which is common in tropical and subtropical countries and has resulted in tremendous economic loss. The pathogenic bacterium R. solanacearum has an extensive host range, and can stay alive in the surroundings of plant roots and soil for a long time. The plants infected with bacterial wilt include more than 300 types in 44 families.

To effectively control bacterial wilt, many efforts have been made internationally in recent years. So far, due to the diversity of the pathogenic bacterium colony and varieties of the host plants, there are few plants developed with bacterial wilt resistance, and the resistance ability, if any, is easily lost. Furthermore, those resistance varieties usually suffer from low product quality and yields, and, as a result, it is difficult to expand the cultivation areas. Graft, though with some success in inhibiting bacterial wilt, requires complex techniques, making the application not economic. Other agricultural control measures such as paddy-glebe rotation are limited by a variety of disadvantages. Pesticides such as streptomycin sulphate and copper fungicide have very limited effects on bacterial wilt control. The function is not consistent and the pathogen bacterium tends to become drug-fast. In summary, so far, no pesticides have been reported to be able to control bacterial wilt effectively.

The pathogenesis of bacterial wilt is as follows: The pathogen bacterium in the soil can intrude the plants through root wounds anytime through the plant life span. Under suitable conditions (such as high temperature and humidity) the pathogenic bacteria penetrates the xylem, multiplies quickly, and clogs the vascular tissues of plants. Accordingly, some control measures should be taken to prevent the bacterial from invasion of roots and colonization within the plants starting from the time of seedling.

Since 1980s, a great amount of research work in controlling of bacterial wilt using avirulent Ralstonia solanacearum has been carried out, which led to many publications. However, most of them remain to be greenhouse experiments and no success in field experiment is reported. Also, the avirulent Ralstonia solanacearum used in the experiment tends to mutate under natural conditions and, accordingly, it is not promising for practical applications.

Other biocontrol agents such as avirulent bacteriocin producer strain (ABPS), Pseudomonas fluorescen, Pseudomonas glumaeand and its variants, Pseudomonas cepacia B5, mutation of Pseudomonas solanacearum, Bacillus spp.B3 and B36, and VAM etc. have been tested for the control of bacterial wilt. They are effective to some extent in greenhouse or seedling stage, but have no effects after 40 days of field planting. Thus, development of an effective biological control agent against plant bacterial wilt disease is imperative.

### SUMMARY OF THE INVENTION

The present invention provides a biological formulation and application method effectively controlling bacterial wilt.

The invention provides a strain of Paenibacillus polymyxa obtained from soil in the suburb of Nanchang city, Jiangxi province of China. The bacterial, its culture broth or the cell-free filtrate of the culture broth are effective in inhibiting bacterial wilt of tomato, green pepper, eggplant and tobacco in field trials. The strain was deposited in China General Microbiological Culture Collection Center (CGMCC) on Oct. 31, 2002, under accession number CGMCC No. 0829.

Therefore, first, this invention provides a strain of Paenibacillus polymyxa HY96-2, under accession number CGMCC No. 0829.

Second, this invention provides a biological formulation for agricultural applications. The formulation contains live microorganisms of Paenibacillus polymyxa or cell-free filtrate from its culture broth. In following examples of field application, the strain of Paenibacillus polymyxa is CGMCC No. 0829.

For a better application, the said biological formulation contains culture broth comprising the live cells of Paenibacillus polymyxa CGMCC No. 0829 and cell-free filtrate of its culture broth.

The terms “culture broth”, “live cells”, “cell-free filtrate” used in the invention have the meanings acceptable to professionals in the field of biotechnology. The said culture broth is obtained through culturing of the said Paenibacillus polymyxa CGMCC No. 0829 (i.e. the biocontrol agent HY96-2 in the following text) under optimized conditions till a certain cell concentration is reached. The said live cells are the microorganisms with surviving ability and obtained through culturing of Paenibacillus polymyxa HY96-2. The cell-free filtrate is the culture broth with biomass removed.

There is no special requirement of the nutrition sources for culturing of the said strain of this invention. The professionals can select suitable carbon, nitrogen and other nutrition sources based on common knowledge of the area. For example, carbon source can be starch, dextrin, glycerine, glucose, sucrose, inositol, mannite etc; Nitrogen source can be peptone, soybean powder, protein powder, meat extract, rice sugar, wheat bran, yeast extract powder, corn paste, ammonium salt and other organic or inorganic nitrogen chemicals. A proper amount of inorganic salts such as sodium chloride, phosphoric salt such as dipotassium hydrogen phosphate/potassium dihydrogen phosphate, ammonium sulfate manganese sulfate, magnesium sulfate, calcium carbonate etc. can be added to the culture medium. Generally, the commonly known culture medium such as LB agar medium, nutrition agar, glucose yeast extract agar and meat extract agar etc. are applicable. The most suitable composition of culture medium is presented in the following examples. However, the professionals of this area should understand that the culture medium of this invention is not restricted to these specific medium formulations.

There are no special requirements of temperature, pH, aeration rate, pressure and mixing for the culturing of the biological agent of this invention as long as the cells grow under the conditions. Soybean oil or anti-foam agents can be used to remove foams of the culture. For better process control, pH should be controlled within the range of 5.5˜7.5; temperature within 25˜35° C., culturing time within 12˜72 hours. The final cell concentration can be from 1×10¹¹ CFU/ml to 1×10¹² CFU/ml. The above control parameters are used as references only for a better process for this invention, the professionals of the area can obtain similar results of biomass, cell-free filtrate and culture broth using suitable control conditions out of the above ranges.

The culture broth or its dilution (with a dilution rate of 10, 100, 1000 times or higher) can directly be applied as the formulation of this invention. The culture broth can be processed with common separation technologies. The live cells, cell-free filtrate and other active ingredients (extracted from the culture broth) can be directly applied. The biological formulations can contain other materials that do not affect the effects of inhibiting bacterial wilt. For example, for the sake of longer shelf life, the culture broth or its dilution can be mixed with suitable carriers without affecting the effects of inhibiting bacterial wilt, then properly dried and processed as different formulations. Therefore, in a better formulation, the said biological formulation contains carrier selected from the following materials: rice chaff powder, cornstalk powder, peatmoss, light calcium carbonate, talcum powder, attapulgite clay/diatomite or their mixtures. The best carrier candidates are rice chaff powder, attapulgite clay and cornstalk powder. All the above carriers are commercially available. The carrier material should be ground to fine granules within the range of 74 μm-1480 μm.

The carriers are mixed with live cell suspension, cell-free filtrate or culture broth with a weight ratio from 1:0.1 to 1:10 and, from 1:0.2 to 1:1.5 for a better result. The drying method can be common technologies of the area, but not restricted to natural drying, vacuum drying, air drying, boiling drying etc. To maintain the high livability of HY96-2 for a longer storage time, the water content of formulation with organic carriers should be controlled under the range of 3-16% (w/w), and 7-16% (w/w) for a better result. The formulation with inorganic carriers should have water content of 3-6% (w/w).

Furthermore, the present invention provides a procedure of applying the biological formulation to the root area of culls infested with pathogens of bacterial wilt.

The above methods of applying biological formulations to plant root area are common technologies of the area. For example, it can be applied as seed or root soaking in the culture broth or its dilutions before transplanting, direct sprinkling of the seedling beds, and drenching of plant root at planting or during cultivation. If the formulations contain carriers, the formulation should be appropriately diluted before application.

With proper experiment, the professionals of the area can determine the best application dosage of the formulation. For example, when using rice chaffpowder as carriers, 1.5 to 4.5 kg/Mu (1 Mu= 1/15 hectare) formulation is a good application dosage.

As demonstrated in the following, the formulation of this invention can be used to inhibit bacterial wilt of tomato, green pepper, eggplant and tobacco in the field test (some control experiments had a disease incidence of 97%) and the control effects reached 70-85% at the end of harvesting.

The formulation of the invention is effective in inhibiting seedling Rhizoctonia, damping off (pythium aphanidermatum), tomato Fusarium wilt, eggplant Fusarium wilt, cucumber Fusarium wilt, Tobacco brown leaf spot (Alternaria Altelnata), Soybean Fusarium root rot (Fusarium orthocreras).

Therefore, the invention is also about the application of the formulation in inhibiting seedling Rhizoctonia, damping off (pythium aphanidermatum), tomato Fusarium wilt, eggplant Fusarium wilt, cucumber Fusarium wilt, Tobacco brown leaf spot (Alternaria Altelnata) and Soybean Fusarium root rot (Fusarium orthocreras).

The formulation of this invention promotes plant growth and yield (as high as 27.5% for tomato) when bacterial wilt does not occur. The formulation of this invention promotes plant growth and yield for other plants such as spinach, amaranth, cowpea and ryegrass; the plant yield can be promoted as high as 18˜25%.

Therefore, this invention provides a formulation and a method promoting plant growth and productivity.

The formulation of this invention is a strong biocontrol agent with the following functions: (1) The formulation effectively inhibits bacterial wilt of tomato, green pepper, egg plant, and tobacco or delays the incidence of the disease; (2) Even in the later stage of harvesting, the formulation still demonstrates control effects as high as 85%, which has not been reported by any other researchers; (3) The formulation obviously promotes the growth of plants infected with bacterial wilt as well as the growth of plants without the incidence of bacterial wilt; (4) The formulation also inhibits plant diseases such as seedling Rhizoctonia, damping off (pythium aphanidermatum), tomato Fusarium wilt, eggplant Fusarium wilt, cucumber Fusarium wilt, Tobacco brown leaf spot (Alternaria Altelnata) and Soybean Fusarium root rot (Fusarium orthocreras) etc.

The other functions and objectives of this invention are clearly explained as follows in detail.

Deposit Information:

The strain of this invention was deposited in the China General Microbiological Culture Collection Center (CGMCC), on Oct. 31, 2002 under accession number CGMCC No. 0829.

### EXAMPLE 1

### Screening of Strain HY96-2

In this example, Ralstonia solanacearum 1 race Th and Ralstonia solanacearum 2 race Tt, Rhizoctonia solani, Tobacco brown leaf spot (Alternaria altelnata), Piricularia oryzae, cucumber Fusarium oxysporum and Soybean Fusarium root rot (Fusarium orthocreras) are used as indicator pathogens.

In suburb of Nanchang city of Jiangxi province, China, healthy and wilted (sick) tomato plants as well as samples of surrounding soils of the roots were collected, and processed as follows as soon as possible.

The soil samples were separated into three groups.

Soils from rhizosphere: the roots were violently shaken and soils dropping off the roots were collected; Soils from rhizospod: After the first procedure, soils still firmly adhering to the roots were washed off and collected; Soils from rhizoplane: The washed roots were cut into small parts. The parts were mixed with quartz sand and water; the mixture was shaken and the soil solution was collected.

The sample strains were screened from the three soil samples according to the following protocol.

Single strain was purified by streak method on improved yeast extract agar (glucose: 1.0%, yeast extract: 0.5%, KH₂PO₄:0.05%, MgSO₄: 0.05%, agar powder: 1.5-1.6%, pH:7.2-7.4). The obtained sample strains were stored in slant cultures for further analysis.

The antagonistic strains were screened as follows.

The first method: the obtained strains were inoculated on Petri dishes of improved yeast extract agar, cultured under 30° C. for 48 hours. The Petri dishes were sterilized using chloroform. Suspension of pathogenic bacterial Th and Tt (10⁸ cfu/ml) were then inoculated to the Petri dishes. After culturing for 12˜24 hours, the diameters of inhibition zone were recorded and the ratio of number of antagonistic strains to that of total separated samples were calculated. All the sample strains with antagonistic effects were stored for further testing.

The second method: 1 ml suspension of pathogenic bacterial Th and Tt (10⁸ cfu/ml) was mixed with 15 ml culture medium at 45-50° C. The mixture was gently shaken and moved to a Petri dish. Five of the separated sample strains were inoculated on each Petri dish and cultured under 30° C. After culturing for 12˜24 hours, the diameters of inhibition zone were recorded and the ratio of number of antagonistic strains to that of total separated samples were calculated. All the sample strains with antagonistic effects were stored for further testing.

The antagonistic effects of sample strains were tested after 10 subcultures. Those still retaining antagonistic abilities were kept and the antagonistic effects of these strains on other pathogens were analyzed.

Results and Analysis

A total of 40 plant samples were collected. Ten healthy and ten wilted (sick) plants were picked from a plot with disease incidence higher than 50%; 10 healthy and 10 wilted (sick) plants were picked from a plot with disease incidence lower than 20%. In the initial screening, 206 antagonistic bacterial strains were obtained, of which 89 were screened from healthy plants (of a plot with disease incidence >40%), 54 from wilted (sick) plants (of a plot with disease incidence >40%), 35 from healthy plants (of a plot with disease incidence <20%), 28 from wilted (sick) plants (of a plot with disease incidence <20%). Of the antagonistic bacterial, 122, 53 and 31 were from rhizospod, rhizosphere, rhizoplane respectively. Through above two screening experiments, 98 strains with antagonistic effects on inhibiting bacterial wilt were obtained.

The obtained 98 antagonistic bacterial strains were sub-cultured 10 times on improved yeast extract slant, with each occurring every 6 days. The antagonistic ability was then measured. Only 49 strains maintained the initial antagonistic abilities, of which, 21, 12 and 16 strains were from rhizoplane, rhizospod and rhizosphere of the original host plants respectively. This indicates that the bacterial strain from rhizoplane had a better chance to be antagonistic. Some strains with antagonistic abilities are presented in Table 1.

TABLE 1
The bacteriostatic action of some obtained strains against
pathogens of bacterial wilt

| | Average Diameter of | |
|---|---|---|
| | Inhibition Zone (mm) | |
| Soil | Tb | Tt |

| Sample | Strains | 1* | 2* | 1* | 2* |
|---|---|---|---|---|---|
| 1-1 | HH-3 | 20.0 | 18.5 | 17.0 | 16.6 |
| | HH-5 | 22.6 | 17.0 | 20.6 | 15.2 |
| 1-2 | HY-2 | 35.2 | 30.8 | 36.8 | 32.6 |
| | HY-14 | 30.4 | 29.2 | 25.5 | 25.8 |
| | HY-22 | 18.4 | 13.2 | 15.2 | 13.6 |
| 2-2 | DY-15 | 25.6 | 20.4 | 27.8 | 22.5 |
| 2-3 | DF-26 | 10.8 | 9.2 | 12.5 | 10.8 |
| 3-1 | HH-34 | 20.4 | 15.6 | 16.5 | 14.3 |
| | HH-42 | 24.7 | 17.8 | 20.6 | 18.2 |
| 3-2 | HY-3 | 30.5 | 25.7 | 32.5 | 30.4 |
| | HY-30 | 17.6 | 12.6 | 12.5 | 11.8 |
| 3-3 | HF-22 | 15.8 | 12.2 | 17.6 | 15.3 |
| 4-1 | DH-16 | 12.5 | 10.5 | 14.2 | 12.9 |
| | DH-18 | 21.9 | 16.8 | 21.2 | 18.8 |
| 4-2 | DY-21 | 16.7 | 13.6 | 18.6 | 16.7 |

1* - Method 1;
2* - Method 2

The antagonistic abilities against other pathogenic strains such as Rhizoctonia solani, Tobacco brown leaf spot (Alternaria altelnata), Piricularia oryzae, cucumber Fusarium oxysporum and Soybean Fusarium root rot (Fusarium orthocreras) etc. were tested, the results are presented in Table 2.

TABLE 2
The fungistatic action of some obtained strains against
pathogenic fungi

| | Width of Inhibition Strips (mm) | | | | |
|---|---|---|---|---|---|
| Pathogenic fungi | HY-2 | HH-3 | HY-14 | DY-15 | HY-3 |
| Rhizoctonia solani | 25.6 | 20.8 | 26.8 | 15.8 | 17.3 |
| Alternaria altelnata | 28.5 | 14.5 | 18.2 | 16.2 | 18.8 |
| Piricularia oryzae | 22.4 | 22.3 | 20.5 | 18.5 | 22.6 |
| Wilt Fusarium | 32.1 | 12.5 | 13.5 | 15.3 | 24.2 |
| orthoceras | | | | | |
| Fusarium | 35.7 | 14.3 | 18.6 | 14.2 | 26.4 |
| orthocreras | | | | | |

The above results demonstrated that strains HY-2, HY-14, HY-3 obtained from tomato rhizosphere had apparent antagonistic function against some pathogenic fungi as well as Th and Tt. This indicated that strain HY-2, HY-14, HY-3 had strong antagonistic function and broad antibiogram. They were named as HY96-2, HY96-14 and HY96-3 respectively.

### EXAMPLE 2

### The Characteristic Analysis of Strain HY96-2

In this section, the characteristics of strain HY96-2 obtained from rhizosphere in Nanchang of China described above was analyzed.

Staining: Gram Staining and Acid Fast Staining

Morphological Character: The strains were cultured on nutrition agar, meat extract agar medium for 48 days under 32° C. The cells were stained and the morphology of the cells was observed under optical microscope. The surface characteristics of the cells were observed under electronic microscope.

Chemical Analysis of Cell walls: the amino acid and carbohydrate of cellular hydrolysate was analyzed by thin layer chromatography.

Culture Characteristics: the strain was cultured on LB agar, nutrition agar, glucose yeast agar and meat extract agar medium for 48-72 days under 32° C. The characteristics and colors of the colonies were observed.

Physiological Characteristics: referring to methods in “Bergey's Manual of Systematic Bacteriology” Vol. II.

16S rDNA sequence data analysis: Total cellular DNA were extracted. The general primers were used to PCR-amplify the 16S rDNA. The PCR product was purified and its sequence was analyzed using “Taq DyeDeoxy Terminator Cycle Sequencing Kit”. Electrophoresis and data analysis were carried out using Applied Biosystems DNA Sequencer (model 377). The sequence data of 16S rDNA were compared with those of related species, genus in GenBank database to determine the classification of this strain.

Results:

(1) HY96-2 was Gram positive, facultatively negative and acid-fast negative.

(2) The cell of HY96-2 had a shape of a straight or near-straight rod. There was an elliptical gemma in a slightly expanded cyst with surrounding flagella. It was aerobic, facultative anaerobic and produced no soluble pigment in nutrition agar medium.

(3) HY96-2 strain contained meso-DAP (diaminopimelic acid) aminoacetic acid, having no characteristic carbohydrate; It had cell wall type II.

(4) The culture characteristic of HY96-2 is presented in Table 3.

TABLE 3
The culture characteristic of strain HY96-2

| Culture Medium | Colony Color and Morphology | |
|---|---|---|
| LB Agar | Taffy Color | Moist Smooth |
| Nutrition Agar | Almond White Color | Viscous |
| Glucose Yeast Agar | Light Yellow White Color | Protuberant, Viscous |
| Meat Extract Agar | Grey White Color | Moist Smooth |

(5) Physiological Characteristics of HY96-2: referring to Table 4.

TABLE 4
The Physiological characteristics of strain HY96-2

| Reactions | Results |
|---|---|
| 6.5% NaCl growth | − |
| Catalytic Reaction | + |
| Oxidase Reaction | + |
| Nitric Salt | + |
| Reduction | |
| V-P Test | + |
| Indole Reaction | − |
| H₂S Reaction | − |
| Citrate Salts Usage | − |
| Gelation | − |
| Liquefaction | |
| Cellulose Growth | − |
| Tween 80 | + |
| 41° C. Growth | + |
| 4° C. Growth | − |
| Starch Hydrolysis | + |
| Urea Hydrolysis | − |
| Lecithine Hydrolysis | − |
| Esculin Hydrolysis | − |
| Acid produced from | |
| carbornihydrate: | |
| Glucose | + |
| L-arabinose | + |
| L-rhamnose | − |
| Fructose | − |
| Xylose | + |
| Mannitol | + |
| Galactose | + |
| Ribose | − |

(6) Strain HY96-2 was congeneric with Paenibacillus and 99% congenial with Paenibacillus polymyxa.

The sequence data analysis of 16S rDNA indicated that HY96-2 was congeneric with Paenibacillus, Gram positive and facultative negative, not acid-fast, in the shape of rod, having gemma with flagella. There was only one gemma in a cyst. All the culture and physiological characteristics were same with those of Paenibacillus polymyxa. Therefore, strain HY96-2 was identified to be Paenibacillus polymyxa. The strain was deposited in the China General Microbiological Culture Collection Center (CGMCC) on Oct. 31, 2002, under accession number CGMCC No. 0829.

### EXAMPLE 3

### The Culture Process of Strain HY96-2

1) Culture in a 5 L Bioreactor

A culture medium consisting of saccharified starch, yeast powder, protein powder, glucose, MgSO₄, KH₂PO₄ and CaCO₃ etc. was added to a 5 liter automatically controlled bioreactor. The system was sterilized at 121° C. for 30 minutes and inoculated with HY96-2 flask cultures. The aeration rate was 0.4˜2; Agitation speed was 300˜800 rpm; temperature was 25˜35° C.; culturing time was 24˜48 hours. The final cell density was 1.37×10¹² cfu/ml.

2) Culture in a 50 L Bioreactor

Same culture medium and conditions of 5 L bioreactor were applied. The final cell density was 2.09×10¹¹ cfu/ml.

3) Culture in a 1000 L Bioreactor

Same culture medium was used. The cultures from a 5 L bioreactor were inoculated into this 1000 L bioreactor. Culture conditions were maintained as those in the 5 L bioreactor except with an aeration rate of 0.4˜1 and agitation speed of 100˜350 rpm. The final cell density was 1.02×10¹¹ cfu/ml.

### EXAMPLE 4

### Formulation of Strain HY96-2 with Different Carriers

1) Rice Chaff Powder as Carriers

Rice chaff powder with granularity of 74-1480 μm was selected. The culture broth and rice chaff powder with granularity of 74-1480μ were thoroughly mixed in a ratio of 0.2-5 (w/w). The mixture were dried using natural, vacuum or boiling bed methods, which resulted in the final products with water contents of 14%, 13.2% and 14.3% respectively.

2) Attapulgite Clay as Carriers

Attapulgite clay with granularity less than 44μ was used. The culture broth was centrifuged and filtered to separate the biomass from cell-free filtrate. The cells were washed several times and diluted to the original culture concentration. The suspension and attapulgite clay powder were mixed thoroughly in a ratio of 3 (w/w). The mixture were dried using natural, vacuum or boiling bed methods, which resulted in the final products with water contents of 4.5%, 4.8% and 4.0% respectively.

3) Cornstalk as Carriers

The cornstalk with granularity of 370˜740μ was used. The filtrate obtained as described in section 2) and cornstalk powder were mixed thoroughly in a ratio of 3 (w/w). The mixture were dried using natural, vacuum or boiling bed methods, which resulted in the final products with water contents of 14.1%, 14.8% and 13.8% respectively.

The characteristics of bacterial in this formulation were analyzed as following: 1 g sample was put into a flask, followed by 10 ml sterilized water. It was then cultured in a shaking bed at 150 rpm for 1 hour. 1 ml of the culture was moved into a test tube with 9 ml sterilized water. The culture was streak inoculated on to a Petri dish with yeast extract medium. The Petri dish was then incubated at 30° C. and the morphology of the colonies were observed from 48th to 72nd hour.

The strain Paenibacillus polymyxa was identified by its culture characteristics. It grew well on a Petri dish of yeast extract; the color and character of the colony was consistent with that of a single colony cultured simultaneously, whose characteristics were in intermediate size, semitransparent, upheaval, smooth-faced, clean-cut edged, shinning, pigment free, highly viscous when picked with a inoculation needle.

The number of live bacterial cells in the formulation was measured by plate count method. 10 g sample of the formulation were moved to a flask with 90 ml water under sterilized conditions; the flask was put on a shaking bed and shaken for 1 hour at 150 rpm. 0.1 ml of each appropriate diluted suspension was moved to a Petri dish of yeast extract medium with 5 replicates. The samples were evenly spread on the surface of agar, and then were cultured at temperature 30° C. for 20˜24 hours. The average number of colonies were counted and calculated as follows:

$$C ( cfu \text{/} g ) = \frac{N \times D}{W}$$

C—cell concentration in the formulation of Paenibacillus polymyxa, cfu/g.
N—average colony counts of 5 replicates
D—total dilution rate
W—sample weight

### EXAMPLE 5

### The Measurement of Toxicity of Biocontrol Agent HY96-2 Against Pathogen of Bacterial Wilt in Greenhouse

In this example, the challenging strains were pathogens of bacterial wilt Ralstonia solanacearum race 1 Th and race 2 Tt. The biocontrol strain was HY96-2.

The measurement of inhibition zone:

The Cup-plate method was used: HY96-2 was inoculated in LB liquid medium and cultured at 120 rpm for 36 hours. The supernatant of the culture broth was filtered through a 0.22 μm sterilized filter and the bacterial-free filtrate was collected. Pathogen of bacterial wilt Th and Tt were spread on LB agar Petri dish and cultured at 30° C. for 48 h. The bacterial concentration was diluted to 10⁹ cfu/ml with 0.85% physiological saline. The mixture of 30 μl of the suspension and 30 ml LB culture medium at 45° C. was added into a Petri dish.

Three Oxford cups were placed on a plate with 20011 bacterial-free suspension each cup, using sterilized water as control. After 46 hours at 8° C., the plate was cultured for 18-24 hours at 30° C. Diameters of the inhibition zones were measured and each test was replicated three times. The results are presented in Table 5.

TABLE 5
The antibacterial effects of HY96-2 metabolites on pathogens of
bacterial wilt

| | Diameter of Inhibition Zone (mm) | | | | |
|---|---|---|---|---|---|
| | | Replication | | | |
| Cell-free Filtrate | Strains | 1 | 2 | 3 | Average |
| Control | Tb | 0 | 0 | 0 | 0 |
| | Tt | 0 | 0 | 0 | 0 |
| Dilution: 1000 | Tb | 10.22 | 9.65 | 10.25 | 10.04 |
| | Tt | 8.98 | 9.82 | 10.09 | 9.63 |
| Dilution: 100 | Tb | 12.62 | 12.78 | 13.57 | 12.99 |
| | Tt | 10.88 | 11.46 | 11.22 | 11.19 |
| Dilution: 10 | Tb | 14.96 | 16.25 | 17.78 | 16.33 |
| | Tt | 14.62 | 15.87 | 15.59 | 15.36 |
| Culture Broth | Tb | 24.25 | 23.68 | 24.03 | 23.99 |
| | Tt | 22.52 | 21.68 | 21.88 | 22.03 |

Pathogens of bacterial wilt race 1 and race 2 were used to test the antibacterial capabilities of HY96-2 cells and its culture broth. The experiment showed that the diameters of inhibition zone were increasing with time. Antibacterial experiments of live cells and the cell-free filtrate were carried out; the inhibition zones were small during the initial days, but became larger within a week; the cell-free filtrate had good antibacterial capabilities as well. This demonstrated that the biocontrol agent HY96-2 produced some unidentified active metabolites, which were responsible in controlling of the growth of pathogens of bacterial wilt. In other words, there should be some active chemicals in the cell-free filtrate and, furthermore, the live cells in the formulation strengthened the antibacterial efficiency.

### EXAMPLE 6

### The Selection of Carriers

This example tested the effects of different carriers on livability of HY96-2 cells in the formulation with different pH and water contents. The strain HY96-2 was cultured on improved meat extract medium for 24 hours; the bacterial cells were then collected through centrifugation and mixed with phosphoric acid buffer of different pH. Within 96 hours, the livability of cells did not show significant changes in the pH range of 6.2˜8.0 under room temperature. The cells were mixed with sterilized rice chaff powder or calcium carbonate powder and the mixtures were formulated with different water contents. The bacterial livability was analyzed in the beginning and after 60 days. It was found that the highest livability of live cells reached 91.2% for rice chaff powder formulation with a water content of 7˜16%; For calcium carbonate powder formulation, the highest value was 82.8% when water content was 4˜6%. Table 6 tabulates the test results of different carriers.

TABLE 6
Effects of carriers on the livability of strain HY96-2 cells
(pH 7.2, water content was 7˜16% for organic carriers, 3˜6% for
inorganic carriers, and storage time was 12 months)

| Carrier | Livability, % |
|---|---|
| Rice chaff Powder | 82.9 |
| Cornstalk Powder | 70.5 |
| Peatmoss | 58.9 |
| Light calcium carbonate | 60.3 |
| Talcum powder | 55.6 |
| Attapulgite Clay | 76.3 |
| Diatomite + Light calcium carbonate | 62.7 |

Table 6 indicated that the livability of strain HY96-2 cells was highest using rice chaff powder as carrier. Therefore, rice chaff powder was chosen as the carrier in the following experiment. Because Paenibacillus polymyxa was highly dry tolerant, it was not sensitive to water content. To optimize the livability of Paenibacillus polymyxa cells for a longer storage period, the water content should be in the range of 7-16%.

### EXAMPLE 7

### Field Application of Strain HY96-2 Culture Broth for Biocontrol of Bacterial Wilt and Yield Promotion

Application method: 2500 ml culture broth (1×10⁸ cfUlml) was used for one Mu.

1^{st} Application: Seed Soaking

100 ml HY96-2 culture broth were diluted 100 times. Seeds enough for one Mu cultivation wrapped in a piece of gauze were soaked in the culture broth for 30 minutes. The seeds then were airing dried and planted in a seedbed. The HY96-2 culture broth was evenly sprinkled to the seedbed.

2nd Application: Nursery Plot Planting

After the seedling was planted in a nursery plot, 200 ml HY96-2 culture broth diluted 500-600 times was evenly sprinkled to the nursery plot for one Mu planting field.

3^{rd} Application: Field Planting

After field planting, 1200 ml HY96-2 culture broth diluted 500-600 times was evenly poured into the root area of each plant in one Mu planting field.

4th Application: 30 Days After Field Planting

After 30 days of field planting, 1000 ml HY96-2 culture broth diluted 600-700 times was evenly poured to the root area of each plant in one mu planting field.

The incidence of disease was surveyed starting from the onset of bacterial wilt. Biocontrol effects and yields were calculated after 80 days of field planting, when was the later stage of harvesting. The results were presented in Table 7.

TABLE 7
Biocontrol of bacterial wilt and yield promotion by strain HY96-
2 culture broth

| | | Days after planting | | | | |
|---|---|---|---|---|---|---|
| | | Incidence | | | Control | Yield |
| | | of Disease (%) | | | Effect | Promotion |
| Plants | | 30 | 50 | 80 | (%) | (%) |
| Tomato | HY96-2 | 2.0 | 2.5 | 15.5 | 83.5 | 252.8 |
| | Streptomycin | 33.0 | 40 | 67.5 | 27.9 | 38.7 |
| | Control | 55.0 | 75 | 93.7 | — | — |
| Green | HY96-2 | 1.8 | 5.2 | 14.7 | 82.2 | 86.8 |
| Pepper | Streptomycin | 10.2 | 17.5 | 52.3 | 36.6 | 42.5 |
| | Control | 19.6 | 25.2 | 82.5 | — | — |
| Egg | HY96-2 | 1.2 | 3.6 | 7.0 | 84.8 | 75.6 |
| Plant | Streptomycin | 15.3 | 28.5 | 30.2 | 34.3 | 30.2 |
| | Control | 20.3 | 34.2 | 46.0 | — | — |

The data in table 7 demonstrated that, after 80 days of field planting (later stage of harvesting), the incidence of tomato bacterial wilt was as high as 93.7%. For tomato, the control effect reached 83% and yield increased 252.8%; for green pepper, the control effect was 82.2% and yield increased 86.8%; for eggplant, the control effect was 84.8% and yield increased 75.6%.

### EXAMPLE 8

### The Biocontrol of Tomato Bacterial Wilt and its Growth Promotion by HY96-2 Formulation in Greenhouse Potted Planting

Tomato cultivar Zhongshu 6, which was susceptible to pathogen infection, was used for the experiment. The seeds were planted in vermiculite. The seedling was moved to potted soils when it had 3-4 leaves.

The formulation prepared according to example 4 was diluted 200 times. The suspension was thoroughly stirred and stayed for 2 hours. The roots of the seedling were soaked in the suspension for 20 rminutes, and then planted in the potted soils, some of which were inoculated. 300 ml of the suspension were added into each pot. 2 million units streptomycin diluted 2000 times was used as a comparison and water was used as control.

Ralstonia solanacearum Th (biovar 1) was purified on TZC Petri dish and cultured on NA Petri dish or slant under 28-30° C. for 48 hours. The colonies were removed and collected to make a suspension with a concentration of 3×10⁸ cfU/ml.

Fine sandy soil and peatmoss were mixed in a ratio of 2. The Th suspension was mixed with the sterilized carrier mixture to make the inoculum density 106 cfu/g. The inoculated soil was then moved to planting pots with diameters of about 15 cm. Two tomato plants, whose roots were treated with biocontrol formulation HY96-2, were planted in each pot followed by adding 300 ml HY96-2 formulation. After planting, the number of plants infected with bacterial wilt and the diseases seriousness (disease grading) were recorded and the index of disease was calculated. The number of plants infected with bacterial wilt and their disease grades were recorded every 10 days (Table 8). After 3 weeks of planting, the plant height, root length and dry or fresh weight of foliage and roots were measured.

TABLE 8
Biocontrol of tomato bacterial wilt by the HY96-2 formulation
in greenhouse potted planting

| | | | | | | | Incidence | | Index | |
|---|---|---|---|---|---|---|---|---|---|---|
| | Days | Number of Wilted plants | | | | | of | Control | of | Control |
| | After | Grade | | | | | disease | Effect | Disease | Effect |
| Formulation | Planting | 0 | 1 | 2 | 3 | 4 | (%) | (%) | (%) | (%) |
| HY96-2 | 10 | 24 | 0 | 0 | 0 | 0 | 0 | — | 0 | — |
| | 20 | 24 | 0 | 0 | 0 | 0 | 0 | 100 | 0 | 100 |
| | 30 | 24 | 0 | 0 | 0 | 0 | 0 | 100 | 0 | 100 |
| | 40 | 22 | 1 | 0 | 1 | 0 | 8.3 | 91.7 | 4.2 | 95.5 |
| | 50 | 20 | 0 | 1 | 1 | 2 | 16.7 | 83.3 | 13.5 | 86.5 |
| Control | 10 | 24 | 0 | 0 | 0 | 0 | 0 | — | 0 | — |
| | 20 | 22 | 1 | 1 | 0 | 0 | 8.3 | — | 3.1 | — |
| | 30 | 15 | 0 | 1 | 3 | 5 | 37.5 | — | 42.7 | — |
| | 40 | 0 | 1 | 1 | 1 | 21 | 100 | — | 93.8 | — |
| | 50 | 0 | 0 | 0 | 0 | 24 | 100 | — | 100 | — |
| Streptomycin | 10 | 24 | 0 | 0 | 0 | 0 | 0 | — | 0 | — |
| Sulphate | 20 | 23 | 1 | 0 | 0 | 0 | 4.2 | 49.4 | 1.1 | 64.5 |
| | 30 | 18 | 0 | 0 | 4 | 2 | 25.0 | 33.3 | 20.8 | 51.3 |
| | 40 | 1 | 0 | 1 | 2 | 20 | 95.8 | 4.2 | 91.7 | 2.2 |
| | 50 | 0 | 0 | 0 | 0 | 24 | 100.0 | 0 | 100.0 | 0 |

The results showed that the HY96-2 formulation can delay the onset of tomato bacterial wilt 20 days. When the incidence of disease for control experiment reached 100%, few plants treated with HY96-2 formulation were infected. The control effect reached 95.5% in the early stage of growth and 86.5% in the later stage of growth. The control effect of streptomycin sulphate was very low and negligible in the later stage of growth.

Treated with HY96-2 formulation, tomato plants grew well in initial three weeks and were much higher than controls thereafter. The results are presented in table 9.

TABLE 9
Growth promotion of tomato by HY96-2 formulation

| | Average Height | | | Average Dry | Dry Weight |
|---|---|---|---|---|---|
| | of Plants (cm) | | | Weight at 6^{th} | Promotion |
| Formulation | 4* | 5* | 6* | Week (g) | (%) |
| HY96-2 | 24.6 | 30.5 | 38.8 | 32.8 | 28.1 |
| Control | 14.4 | 20.5 | 30.9 | 25.6 | — |
| Streptomycin | 14.3 | 21.2 | 29.8 | 25.3 | −1.1 |
| Sulphate | | | | | |

*weeks after planting

The data in table 9 showed that HY96-2 formulation could significantly promote the growth of tomato plants, especially in the early stage of growth. In the 5^{th} week after planting, the height of plants treated with the formulation was 10 cm higher than the control. In the 6^{th} week, the dry weight of treated plants increased 28%.

### EXAMPLE 9

### The Biocontrol of Bacterial Wilt using HY96-2 Formulation in Field Test

The formulation was tested to control the bacterial wilt of tobacco, tomato, eggplant and green pepper in field trials. The control effects on bacterial wilt and yield promotion were systematically studied. The field tests in three consecutive years demonstrated consistent control effects on bacterial wilts of tomato, green pepper, eggplant and tobacco. Some of the results are presented as following, in which the incidence of disease was surveyed periodically and the control effect at the last time of surveying (later stage of harvesting) was calculated.

Application Dosage: 3000 g/Mu

1 st Application: Seeds Soaking

50 g formulation diluted 300 times was thoroughly stirred and let stayed for 2 hours. Seeds wrapped in a piece of gauze (enough for one Mu planting) were soaked in the prepared suspension for 30 minutes. After the seeds were dried by airing, they were planted in a seeding bed enough for one Mu field planting and the suspension was evenly sprinkled to the seeding bed.

2^{nd} Application: Nursery Pot Planting

200 g formulation diluted 500-600 times was thoroughly stirred and let stayed for 2 hours. After the seedling was planted in nursery pots, the suspension was evenly sprinkled to the nursery pots for one Mu field planting.

3^{rd} Application: Field Planting

1500 g formulation diluted 500-600 times was thoroughly stirred and let stayed for 2 hours. After the seedling was planted, the suspension was evenly sprinkled to the root area of the plants.

4^{th} Application: 30 Days After Field Planting

1250 g formulation diluted 600-700 times was thoroughly stirred and let stayed for 2 hours. After 30 days of field planting, the suspension was evenly sprinkled to the root area of the plants.

Each field experiment had four replications; the total number of experiment plots was 20. Results were presented in table 10˜13.

TABLE 10
Biocontrol of tomato bacterial wilt by HY96-2 formulation in field trials

| | | | 35* | | | 60* | | | 90* | | | |
|---|---|---|---|---|---|---|---|---|---|---|---|---|
| | | | | Index | | | Index | | | Index | | Yield |
| Formulation | | Total | Total | of | Control | Total | of | Control | Total | of | Control | Promotion |
| (g/Mu) | Replication | plants | Culls | Disease | Effect % | Culls | Disease | Effect % | Culls | Disease | Effect % | (%) |
| HY96-2 | 1 | 100 | 0 | 0 | 100 | 14 | 2.89 | 96.09 | 16 | 15.07 | 84.63 | 300.83 |
| Formulation | 2 | 100 | 0 | 0 | 100 | 10 | 2.78 | 96.46 | 18 | 16.61 | 81.87 | |
| (3000) | 3 | 100 | 0 | 0 | 100 | 8 | 2.22 | 97.02 | 20 | 18.65 | 80.08 | |
| | 4 | 100 | 0 | 0 | 100 | 9 | 2.30 | 97.09 | 19 | 16.38 | 83.31 | |
| | X | | | 0 | 100 | | 2.55 | 96.67 | | 16.68 | 82.47 | |
| Streptomycin | 1 | 100 | 30 | 3.11 | 67.06 | 36 | 20.56 | 72.17 | 59 | 58.03 | 40.80 | 53.40 |
| (240) | 2 | 100 | 25 | 3.22 | 67.80 | 35 | 20.44 | 73.94 | 58 | 57.62 | 37.10 | |
| | 3 | 100 | 19 | 3.11 | 70.55 | 33 | 20.89 | 71.98 | 66 | 65.68 | 29.85 | |
| | 4 | 100 | 25 | 3.30 | 64.90 | 36 | 20.50 | 74.05 | 71 | 61.35 | 37.48 | |
| | X | | | 3.19 | 67.58 | | 20.60 | 73.04 | | 60.67 | 36.31 | |
| Control | 1 | 100 | 50 | 9.44 | 0 | 95 | 73.89 | 0 | 99 | 98.03 | 0 | Yield per |
| | 2 | 100 | 48 | 10.00 | 0 | 88 | 78.44 | 0 | 94 | 91.61 | 0 | Mu |
| | 3 | 100 | 55 | 10.56 | 0 | 83 | 74.56 | 0 | 95 | 93.63 | 0 | 721 Kg |
| | 4 | 100 | 50 | 9.40 | 0 | 100 | 79.00 | 0 | 100 | 98.13 | 0 | |
| | X | | 50.8 | 9.85 | 0 | 91.5 | 76.46 | 0 | 97 | 95.35 | 0 | |

*Days after field planting

The results in table 10 showed that, when 3.0 kg/Mu of the formulation was applied to tomato, the control effect reached 96.67% after 60 days of field planting. After 90 days of field planting (later stage of harvesting), the control effect reached 82.47% and yield promotion reached 300.83% when the incidence of disease of control was as high as 97%.

TABLE 11
Biocontrol of green pepper bacterial wilt by HY96-2
formulation in field trials

| | Days* | | | | | | |
|---|---|---|---|---|---|---|---|
| | 40 | | 60 | | 110 | | |
| | Index of | Control | Index of | Control | Index of | Control | Yield |
| | Disease | Effect | Disease | Effect | Disease | Effect | Promotion |
| Formulation | (%) | (%) | (%) | (%) | (%) | (%) | (%) |
| HY96-2 | 0 | 100 | 0.14 | 98.43 | 2.58 | 79.23 | 147.9 |
| 2 kg/Mu | | | | | | | |
| Streptomycin | 0.38 | 74.01 | 6.33 | 44.49 | 7.80 | 37.08 | 37.30 |
| 0.24 kg/Mu | | | | | | | |
| Control | 1.47 | — | 11.41 | — | 12.40 | — | — |

*Days after field planting

The results in table 11 showed that the control effect reached 79.23% and yield increased 147.9% when 2 kg/Mu HY96-2 formulation was applied to green pepper.

TABLE 12
Biocontrol of eggplant bacterial wilt by HY96-2 formulation in
field trials

| | Days* | | | | | | |
|---|---|---|---|---|---|---|---|
| | 30 | | 60 | | 90 | | |
| | Index | | Index of | | Index of | Control | Yield |
| | of | Control | Disease | Control | Disease | Effect | Promotion |
| Formulation | Disease(%) | Effect(%) | (%) | Effect(%) | (%) | (%) | (%) |
| HY96-2 | 0.11 | 98.95 | 1.50 | 96.43 | 6.43 | 85.74 | 166.6 |
| 3 kg/Mu | | | | | | | |
| Streptomycin | 3.75 | 63.45 | 9.48 | 77.39 | 25.05 | 44.63 | 28.80 |
| 0.24 kg/Mu | | | | | | | |
| Control | 10.25 | — | 41.93 | — | 45.25 | — | |

*Days after field planting

The results in table 12 showed that, when 3 kg/Mu HY96-2 formulation was applied to eggplant, the control effect reached 85.74% and yield increased 166.6%.

TABLE 13
Biocontrol of tobacco bacterial wilt by HY96-2 formulation in
field trials

Days*
110

| | Incidence of | Index of Disease | Control Effect |
|---|---|---|---|
| Formulation | Disease (%) | (%) | (%) |
| HY96-2 | 52.0 | 20.9 | 67.4 |
| 3.5 kg/Mu | | | |
| Streptomycin | 62.0 | 26.3 | 58.9 |
| Control | 76.3 | 64.1 | — |

*Days after field planting

The results in table 13 showed that, when 3.5 kg/Mu HY96-2 formulation was applied to tobacco, the control effect reached 67% after 110 days of field planting.

### EXAMPLE 10

### Biocontrol of Tomato Bacterial Wilt by Formulations of Culture Broth, Live Cells and Cell-Free Filtrate

According to the description 1), 2), 3) in example 4, part of the HY96-2 culture broth obtained under culture conditions of example 3 was used directly to make “culture broth formulation”. The live cells were used to make “live cells formulation”; the cell-free filtrate was used to make “cell-free filtrate formulation”.

The field trial procedures of were as same as that of example 9 and 2.5 kg/Mu formulation was applied. Results were presented in table 14

TABLE 14
Biocontrol of tomato bacterial wilt by formulations of live
cells, culture broth and cell-free filtrate in field trials

| | Days* | | | | | |
|---|---|---|---|---|---|---|
| | 40 | | 70 | | 90 | |
| | Disease | Control | Disease | Control | Disease | Control |
| | Index | Effect | Index | Effect | Index | Effect |
| FormulaTions | (%) | (%) | (%) | (%) | (%) | (%) |
| Broth | 0 | 100 | 0.93 | 97.68 | 13.16 | 83.57 |
| Live Cells | 0 | 100 | 1.67 | 95.83 | 14.61 | 81.76 |
| Cell-free | 0 | 100 | 1.30 | 96.36 | 14.15 | 82.33 |
| Filtrate | | | | | | |
| Streptomycin | 3.52 | 20.70 | 17.59 | 50.78 | 55.19 | 31.09 |
| Control | 4.81 | — | 40.00 | — | 80.09 | — |

*Days after planting

It was clear that formulations of either live cells or cell-free filtrate functioned well in controlling of bacterial wilt. They showed similar effects with culture broth formulation.

### EXAMPLE 11

### Biocontrol of Fungus Diseases by HY96-2 Formulations of Culture Broth, Live Cells and Cell-Free Filtrate in Field Test

In experiment of example 9, it was noticed that the formulation was effective in biocontrol of fungus diseases.

In Hexian, Anhei province, it was found that the formulation was effective controlling seedling Rhizoctonia, damping off and cucumber Fusarium wilt, with control effect being 85% or higher. In Nanchang, Jingxi province, the results showed that the formulation had good controlling effects on tomato Fusarium wilt and eggplant Fusarium wilt, with control effect of 83% or higher. In Jiangle, Fujian province, it was found that the formulation was effective controlling Tobacco brown leaf spot (Alternaria altelnata) with a control effect of 83% or higher. In Heilongjiang province, it showed the formulation was effective inhibiting Soybean Fusarium root rot (Fusarium orthocreras) with a control effect of 83% or higher. The results were presented in table 15.

TABLE 15
Biocontrol effects of HY96-2 formulation on some plant fungus
diseases

| | | Rate of | |
|---|---|---|---|
| | | incidence | Control |
| Disease | Formulation | (%) | Effect (%) |
| Tomato Rhizoctonia solani | Broth | 3.2 | 91.7 |
| | Live Cells | 4.8 | 87.5 |
| | Cell-free | 4.6 | 88.1 |
| | Filtrate | | |
| | Control | 38.5 | — |
| Tomato damping off (pythium | Broth | 3.8 | 87.9 |
| aphanidermatum) | Live Cells | 4.2 | 86.7 |
| | Cell-free | 4.8 | 84.8 |
| | Filtrate | | |
| | Control | 31.5 | — |
| Tomato Fusarium wilt | Broth | 5.15 | 85.2 |
| | Live Cells | 5.45 | 84.3 |
| | Cell-free | 5.82 | 83.3 |
| | Filtrate | | |
| | Control | 34.8 | — |
| Eggplant Fusarium wilt | Broth | 4.62 | 85.8 |
| | Live Cells | 5.0 | 84.6 |
| | Cell-free | 4.76 | 85.4 |
| | Filtrate | | |
| | Control | 32.5 | — |
| Cucumber Fusarium wilt | Broth | 2.1 | 92.7 |
| | Live Cells | 2.6 | 90.9 |
| | Cell-free | 2.4 | 91.7 |
| | Filtrate | | |
| | Control | 28.8 | — |
| Tobacco brown leaf spot | Broth | 10.5 | 84.4 |
| (Alternaria Altelnata) | Live Cells | 11.2 | 83.3 |
| | Cell-free | 11.5 | 82.9 |
| | Filtrate | | |
| | Control | 67.2 | — |
| Soybean Fusarium root rot | Broth | 5.3 | 85.5 |
| (Fusarium orthocreras) | Live Cells | 5.8 | 84.2 |
| | Cell-free | 6.1 | 83.3 |
| | Filtrate | | |
| | Control | 36.6 | — |

It clearly showed that formulations of either live cells or cell-free filtrate functioned well in controlling of Soybean Fusarium root rot (Fusarium orthocreras), tomato Rhizoctonia solani, tomato damping off (pythium aphanidermatum, tomato Fusarium wilt, eggplant Fusarium wilt, cucumber Fusarium wilt and Tobacco brown leaf spot (Alternaria Altelnata). They demonstrated similar effects with culture broth formulation.

### EXAMPLE 12

### The Growth Promotion of Some Plants by HY96-2 Formulation of Culture Broth, Live Cells and Cell-Free Filtrate in Field Tests

In the field trials of different formulations for biocontrol of bacterial wilt, the apparent growth promotion of tomato, green peppers and peanuts were observed. The formulation had apparent growth promotion to non-host as well as host crops of bacterial wilt pathogens. Experiments showed that, when applying HY96-2 formulation to tomato plants not infected with the pathogens of bacterial wilt, the yield increased 27.5%, which mainly occurred in the early period of production (Table 16). Other field experiments showed that the HY96-2 formulation promoted the yields of spinach, amaranth, cowpea and ryegrass as high as 8.3%, 25.0%, 18.7% and 11.9% respectively (Table 17 and 18).

TABLE 16
The yield promotion of tomato by HY96-2 formulation without
the incidence of bacterial wilt

| | Time and yield (Kg) | | | | | | | | | Total | Per Mu | Promotion |
|---|---|---|---|---|---|---|---|---|---|---|---|---|
| Formulation | 10/26 | 10/28 | 10/31 | 11/3 | 11/6 | 11/9 | 11/12 | 11/15 | 11/19 | (Kg) | (Kg) | (%) |
| Control | 0.3 | 2.0 | 3.3 | 0.8 | 0.6 | 1.2 | 1.4 | 2.2 | 2.0 | 13.8 | 2760 | — |
| Broth | 3.3 | 4.1 | 3.9 | 1.0 | 0.6 | 1.5 | 1.2 | 2.0 | 0 | 17.6 | 3520 | 27.5 |
| Live Cells | 3.1 | 3.6 | 3.5 | 1.1 | 0.7 | 1.3 | 1.4 | 2.0 | 0.2 | 16.9 | 3380 | 22.5 |
| Cell-free | 2.8 | 4.0 | 4.0 | 0.8 | 0.6 | 1.4 | 1.2 | 2.2 | 0.4 | 17.4 | 3480 | 26.1 |
| filtrate | | | | | | | | | | | | |

TABLE 17
The yield promotion of amaranth, cowpea by the HY96-2
formulation

| | Amaranth | | | |
|---|---|---|---|---|
| | | Average | Cowpea | |
| | Plant | Fresh | Plant Height | Average Fresh |
| Formulation | Height (cm) | Weight (g) | (cm) | Weight (g) |
| Control | 16.6 | 6.0 | 47.3 | 30.0 |
| Broth | 18.1 | 6.5 | 52.2 | 35.6 |
| Live Cells | 18.0 | 6.2 | 50.8 | 35.2 |
| Cell-free | 17.8 | 6.5 | 51.7 | 35.7 |
| filtrate | | | | |

TABLE 18
The yield promotion of spinach by the HY96-2 formulation

| | | Foliage | Foliage | Root | Root | |
|---|---|---|---|---|---|---|
| | Average | Fresh | Dry | Fresh | Dry | |
| | Height | Weight | Weight | Weight | Weight | Promotion |
| Formulation | (cm) | (g) | (g) | (g) | (g) | (%) |
| Control | 12.73 | 26.59 | 3.46 | 0.74 | 0.15 | — |
| Broth | 11.35 | 33.23 | 3.70 | 0.94 | 0.17 | 25.0 |
| Live Cells | 12.68 | 32.56 | 3.68 | 1.12 | 0.16 | 22.5 |
| Cell-free | 12.92 | 33.18 | 3.62 | 0.92 | 0.17 | 24.8 |
| filtrate | | | | | | |

The results in Table 16-18 indicated that both of the live cells and the cell-free filtrate had apparent growth promotion effects on tomato, spinach, amaranth and cowpea, and the functions were similar to those of culture broth formulation.

Although many examples were presented above, it is self-evident that the invention is applicable under other different conditions, which were acceptable to professionals of the area. Therefore, all variations of this invention are covered by this claim.

What is claimed is:

1. A strain of Paenibacillus polymyxa HY96-2 is deposited at China General Microbiological Culture Collection Center (CGMCC) on Oct. 31, 2002, under accession number CGMCC No. 0829.

2. A biological formulation containing the strain of claim 1 or cell-free filtrates of its culture broth.

3. The biological formulation of claim 2, containing the culture broth of claim 1.

4. The biological formulation of claim 2, containing carriers selected from rice chaff powder, cornstalk powder, peatmoss, calcium carbonate powder, talcum powder, attapulgite clay and diatomite or their mixtures.

5. The biological formulation of claim 4, containing a carrier selected from rice chaff powder, attapulgite clay and cornstalk powder.

6. The biological formulation of claim 5, containing 3˜16% (w/w) water.

7. A method of inhibiting bacterial wilt providing a procedure of applying the formulation of claim 2 to the surroundings of plant roots infected with bacterial wilt.

8. The method of claim 7 wherein said plants are selected from a group of tomato, green pepper, egg plant, and tobacco.

9. The formulation of claim 2 wherein said formulation is effective in inhibiting plant diseases including seedling Rhizoctonia, damping off (Pythium aphanidermatum), tomato Fusarium wilt, eggplant Fusarium wilt, cucumber Fusarium wilt, Tobacco brown leaf spot (Alternaria altelnata) and Soybean Fusarium root rot (Fusarium orthocreras).

10. The formulation of claim 2 wherein said formulation is effective in promoting cell growth and plant yields.
